(19) United States
(12) Patent Application Publication
(10) Pub. No.: US 2010/0241025 A1
(43) Pub. Date: Sep. 23, 2010

(54) COLD REFLEX TESTER

(76) Inventors: Young Min BAE, Seongnam-si (KR); Uk KANG, Ansan-si (KR); Sergey KIM, Ansan-si (KR); Guang Hoon KIM, Suyeong-gu (KR); Dae Sic LEE, Ansan-si (KR)

Correspondence Address: FROMMER LAWRENCE & HAUG LLP, 745 FIFTH AVENUE, NEW YORK, NY, 10151, US

(21) Appl. No.: 12/502,566

(22) Filed: Jul. 14, 2009

(30) Foreign Application Priority Data
Mar. 19, 2009 (KR) 10-2009-0023559

(51) Int. Cl. A61B 5/00

(52) U.S. Cl. 600/555

(57) ABSTRACT

The present invention provides a cold reflex tester, in which an electrical device capable of controlling the temperature and maintaining a fixed temperature at a low level based on an electrical signal from a controller is used in a cold stimulator that is brought into contact with a subject's body as a means for applying a cold stimulus to the subject's body. Thus, it is possible to apply a more constant stimulus to a subject's body during measurement and thus improve the reliability of the measurement.

The cold reflex tester of the present invention may include a cold stimulator for applying a cold stimulus to the subject's body being in contact with the cold stimulator by maintaining a fixed low temperature based on an electrical signal applied from a controller, a blood flowmeter for measuring blood flow velocity of the subject to detect the body's reaction against the cold stimulus; the controller for controlling the entire system including the cold stimulator and the blood flowmeter and obtaining a measurement result from the blood flowmeter; and a display for displaying the measurement result received from the controller on a screen.

### CROSS-REFERENCE TO RELATED APPLICATION

This application claims the benefit of Korean Patent Application No. 10-2009-0023559, filed on Mar. 19, 2009, the entire disclosure of which is hereby incorporated by reference.

### BACKGROUND OF THE INVENTION

1. Field of the Invention

The present invention relates to a cold reflex tester, and more particularly, to a cold reflex tester configured to automate a cardiovascular autonomic function test against a cold stimulus.

2. Description of Related Art

Sympathetic nerves of the cardiovascular system can be examined by stimulating the rostral ventrolateral medulla, a part of the brainstem reticular formation as the vasomotor center, by applying a cold stimulus to a body to activate the sympathetic nervous system, detecting a measurement index of blood circulation against the cold stimulus, and then comparing the detected value with data of normal persons.

This test method is called a cold reflex test, and typically a cold pressor test has been developed and used. The cold pressor test that uses the functions of circulatory organs is widely used in the diagnosis of autonomic nervous system in traditional Oriental medicine and is related to an endothelial function test in Western medicine.

The general process of the cold pressor test is as follows:

(1) Stabilizing a subject and measuring blood pressure at predetermined time intervals;

(2) Dipping the subject's hand in cold water for about 1 minute;

(3) Measuring blood pressure at predetermined time intervals while the subject's hand is dipped in cold water; and

(4) Measuring blood pressure at predetermined time intervals after the subject's hand is removed from the cold water.

However, the conventional cold pressor test is performed to measure the change in blood pressure according to the cold water stimulus, and it has been difficult to develop an automated medical device due to difficulties in maintaining a constant stimulus and monitoring the measurement of blood pressure.

Especially, it is impossible to maintain the temperature of cold water, used as an external stimulus, constant during each test. Moreover, the measurement of blood pressure at regular intervals is carried out manually by a test operator, and it is difficult to monitor the change in the body against the cold stimulus in real time.

Thus, it is necessary to develop a cold pressor tester (clod reflex tester) as an automated medical device for testing the autonomic nervous function of human body against a cold stimulus.

### SUMMARY OF THE INVENTION

The present invention has been made in an effort to solve the above-described problems associated with prior art. Accordingly, the present invention provides an automated cold reflex tester, which can maintain a cold stimulus constant and measure and monitor the body's reaction against the cold stimulus in real time.

In one aspect, the present invention provides a cold reflex tester, which applies a cold stimulus to a subject's body and detects and monitors the body's reaction against the cold stimulus, the cold reflex tester including: a cold stimulator for applying a cold stimulus to the subject's body being in contact with the cold stimulator by maintaining a fixed low temperature based on an electrical signal applied from a controller; a blood flowmeter for measuring blood flow velocity of the subject to detect the body's reaction against the cold stimulus; the controller for controlling the entire system including the cold stimulator and the blood flowmeter and obtaining a measurement result from the blood flowmeter; and a display for displaying the measurement result received from the controller on a screen.

The cold stimulator may include a Peltier module controlled by an electrical signal applied from the controller.

The cold stimulator may include a heat sink mounted on a high temperature region of the Peltier module, a cooling fan for cooling the heat sink, and a contact region, with which the subject's body is brought into contact, formed on a low temperature region of the Peltier module.

The cold reflex tester of the present invention may further include a touch sensor mounted on the surface of the cold stimulator to detect a touch of the body and a release of the touch and output an electrical signal to the controller.

The controller may recognize and record stimulation start and end times based on the electrical signal input from the touch sensor.

The controller may display the stimulation start and end times on the display.

A temperature sensor may be provided in the cold stimulator such that the controller receives a signal of the temperature sensor to control the operation of the cold stimulator.

The blood flowmeter may be a blood flowmeter using the Doppler effect that measures blood flow velocity by transmitting ultrasonic waves to the body and detecting the frequency of the ultrasonic waves reflected from blood vessels.

### BRIEF DESCRIPTION OF THE DRAWINGS

The above and other features of the present invention will be described with reference to certain exemplary embodiments thereof illustrated the attached drawings in which:

FIG. 1 is a diagram showing a configuration of a cold reflex tester in accordance with a preferred embodiment of the present invention;

FIG. 2 is a diagram showing an example of a configuration of a cold stimulator in the cold reflex tester in accordance with the present invention;

FIG. 3 is a diagram showing an example of a configuration of a blood flowmeter in the cold reflex tester in accordance with the present invention; and

FIG. 4 is a graph showing a change in blood flow velocity measured by the blood flowmeter in the cold reflex tester in accordance with the present invention.

### DETAILED DESCRIPTION OF THE INVENTION

Hereinafter, preferred embodiments of the present invention will be described in detail with reference to the accompanying drawings. The features and advantages of the present invention will become more fully understood from the following detailed description with reference to the accompanying drawings. Terms or words used in the specification and claims should be interpreted as meanings and concepts that conform to the technical spirit of the invention, based on the principle that an inventor can properly define the concepts of terms to explain the invention in the best way.

The present invention provides a cold reflex tester configured to automate a cardiovascular autonomic function test against a cold stimulus, in which a cold stimulator capable of controlling the temperature based on a control signal applied from a controller and maintaining a fixed low temperature is used instead of cold water conventionally used as an external cold stimulus. Moreover, a method of measuring blood flow velocity is employed to facilitate the measurement of the body's reaction against the external cold stimulus.

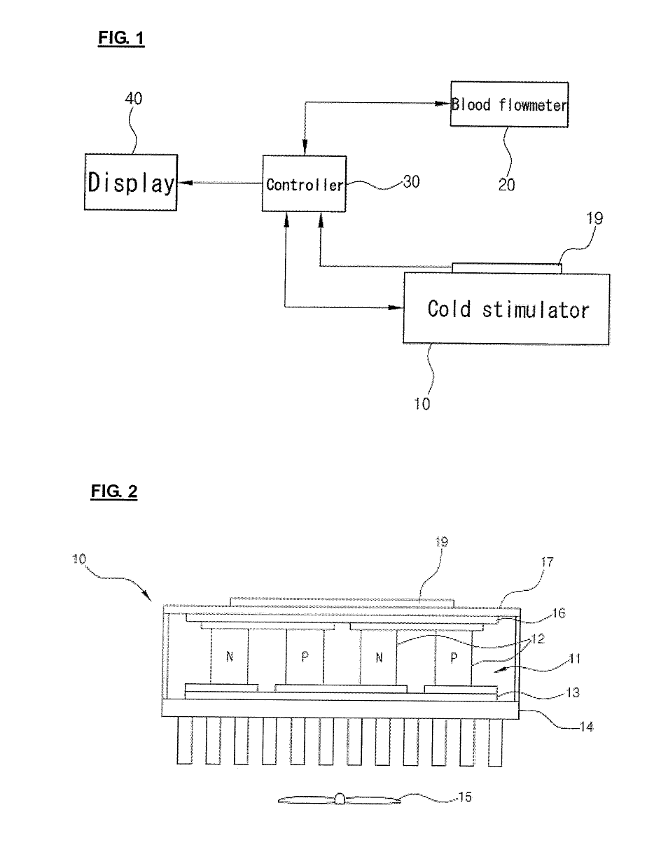

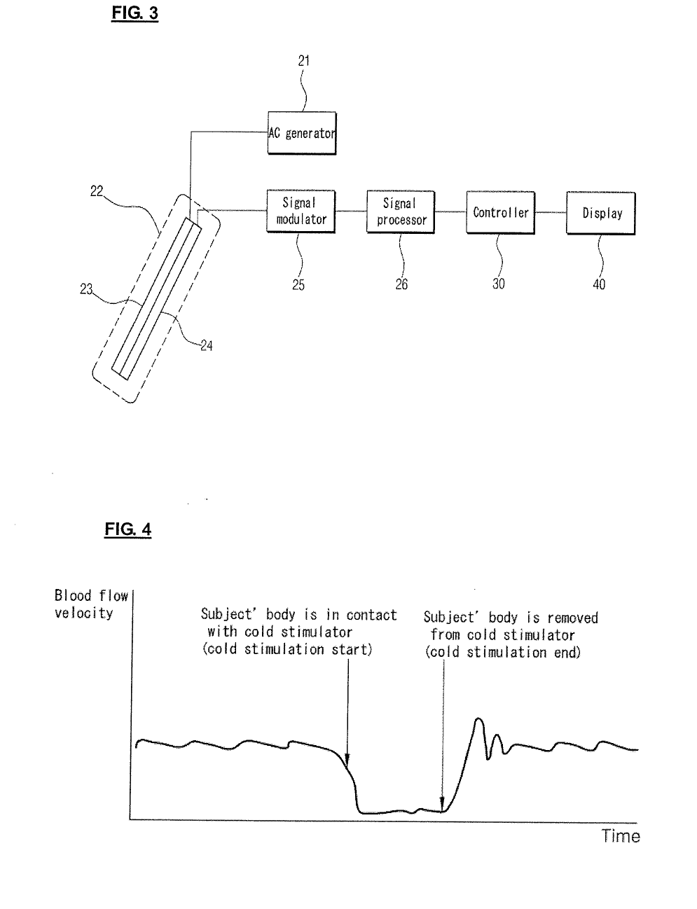

FIG. 1 is a diagram showing a configuration of a cold reflex tester in accordance with a preferred embodiment of the present invention, FIG. 2 is a diagram showing an example of a configuration of a cold stimulator 10 in the cold reflex tester in accordance with the present invention, and FIG. 3 is a diagram showing an example of a configuration of a blood flowmeter 20 in the cold reflex tester in accordance with the present invention.

As shown in the figures, the cold reflex tester of the present invention includes a cold stimulator 10, a blood flowmeter 20, a controller 30, and a display 40. The operation of the cold stimulator 10 is controlled by an electrical signal applied from the controller 30 such that the temperature can be controlled and a fixed low temperature can be maintained. When the cold stimulator 10 is brought into contact with a subject's body, it applies a cold stimulus to the subject. The blood flowmeter 20 measure blood flow velocity of the subject to detect the body's reaction against the cold stimulus. The controller 30 controls the entire system including the cold stimulator 10 and the blood flowmeter 20 and obtains a measurement result of the blood flow velocity from the blood flowmeter 20. The display 40 receives the measurement result of the blood flowmeter 20 from the controller 30 and displays the result on a screen.

The cold stimulator 10 is a component element that maintains a fixed low temperature (e.g., 15° C.) to apply a constant cold stimulus to the subject's body (i.e., stimulated region) that is in contact with the cold stimulator 10 during the measurement. The cold stimulator 10 is configured such that the temperature can be controlled by an electrical signal, i.e., a control signal for controlling the operation thereof, applied from the controller 30.

In a preferred embodiment, the operation of the cold stimulator 10 may be controlled by an electrical signal of the controller 30 such that the temperature of a specific region can be controlled or may have a configuration in which a Peltier module 11 capable of maintaining the temperature of a specific region constant at a low level is employed.

As well known in the art, the Peltier module 11 includes a thermoelectric semiconductor device (Peltier element), which is cooled or heated by direct current, and has been widely used in various industrial fields where it is necessary to cool or heat a contact object.

Since the Peltier module, which can cool or heat the contact object by appropriately receiving the direct current required for the operation (corresponding to the control signal for controlling the operation) from the controller, a detailed circuit configuration of its driver, an electrical connection relationship between the Peltier module and the driver, and an electrical connection relationship between the Peltier module and the controller are well-known techniques, detailed description thereof will be omitted.

In general, since the direct current is applied to the Peltier module, a temperature difference occurs on both sides of the thermoelectric semiconductor device. In this case, in the thermoelectric semiconductor device, since a low temperature region absorbs heat and a high temperature region emits heat, the heat is transferred from the low temperature region to the high temperature region, thus operating as a heat pump.

Moreover, it is possible to change the direction in which heat flows by simply reversing the polarity of the current, and it is also possible to change the amount of transferred heat by simply changing the intensity of the current. Thus, the Peltier module can easily cool or heat the contact object.

Especially, the Peltier module has advantages of small size, light weight, versatile shape, easy control of the temperature at room temperature, and good temperature response.

Moreover, since it has no operating part, there is no vibration and noise. Furthermore, since it has no mechanical part fatigued and damaged, it has high reliability and long durability. In addition, since it includes only an electrical wiring, its handling is simple.

The configuration of the cold simulator 10 employing the Peltier module 11 will be described in detail with reference to FIG. 2 below. The cold stimulator 10 may include the Peltier module 11, a heat sink 14 mounted on a high temperature region 13 of the Peltier module 11, and a cooling fan 15 for cooling the heat sink 14 to improve the heat dissipation of the heat sink 14.

A contact region 17, which will be brought into contact with a subject's body to apply a cold stimulus thereto, is formed on a low temperature region 16 of the Peltier module 11. The contact region 17 may be formed by attaching a metal plate or block to the low temperature region 16 of the Peltier module 11 or by coating a portion including the low temperature region 16 with polymer.

Since the driver for driving the Peltier module is a well-known component, it is not shown in FIG. 2.

Although the cold stimulator 10 that employs the Peltier module 11 has been described as above, the present invention is not limited thereto, and the cold stimulator 10 may be formed by employing any known thermostat means capable of maintaining the temperature constant at a low level based on an electrical signal applied from the controller 30.

Moreover, a temperature sensor (not shown) for detecting the temperature of the cold stimulator 10 may be provided to precisely control the temperature of the Peltier module 11 or the thermostat means. The temperature sensor may be placed in a position that is not in direct contact with the subject's body, e.g., in the inside of the contact region 17 or between the contact region 17 and the low temperature region 16.

In a preferred embodiment, a touch sensor 19 may be mounted on the surface that is in direct contact with the subject's body, i.e., on the surface of the contact region 17 of the cold stimulator 10.

The touch sensor 19 is configured to detect a touch of the body and a release of the touch and output an electrical signal corresponding to the detected value to the controller 30.

Thus, the controller 30 can recognize that the subject's body is in contact with the cold stimulator 10 or that the contact is released based on the electrical signal output from the touch sensor 19, and recognize and record stimulation start and end times.

At the same time, the controller 30 can display the stimulation start and end times on a screen of the display 40.

Meanwhile, the blood flowmeter 20 is a component element that measures blood flow of the subject. Conventionally, a hemodynamometer detects the body's reaction against the cold stimulus by measuring blood pressure in the cold pressor test. However, in the present invention, the blood flow velocity of the peripheral circulation such as hand or foot is measured using the blood flowmeter 20, thus detecting the body's reaction against the cold stimulus. The blood pressure and blood flow velocity are the homeostatic functions of the body and have a very high correlation.

In the present invention, the blood flowmeter 20 should be able to non-invasively measure the blood flow velocity from the outside of the body in real time, and it is preferable that a blood flowmeter using the Doppler effect be used.

A Doppler ultrasonic flowmeter can measure the blood flow velocity by transmitting ultrasonic waves having a specific frequency to the body using an ultrasonic transducer and detecting the frequency of the ultrasonic waves reflected from blood vessels, more explicitly, by red blood cells flowing through blood vessels.

An ultrasound blood flowmeter using the Doppler effect disclosed in Korean Patent Application No. 10-2007-0101187 or 10-2008-0110975 filed by the present applicant may be used as the blood flowmeter.

FIG. 3 shows an example of a configuration in which the blood flowmeter 20 disclosed in Korean Patent Application No. 10-2008-0110975 is employed. The blood flowmeter 20 of FIG. 3 includes a measurement unit 22 including an ultrasonic transmitter 23 and an ultrasonic receiver 24, and an alternating current (AC) generator 21 electrically connected to the ultrasonic transmitter 23. Especially, the ultrasonic receiver 24 is sequentially connected to a signal modulator 25, a signal processor 26, the controller 30, and the display 40 such that an electrical signal can be transmitted in that order.

The ultrasonic transmitter 23 receives a signal output from the AC generator 21 and outputs ultrasonic waves corresponding to the received signal, and the AC generator 21 generates AC power for operating the ultrasonic transmitter 23.

During measurement, after the measurement unit 22 including the ultrasonic transmitter 23 and the ultrasonic receiver 24 is brought into contact with a subject's body, the ultrasonic transmitter 23 receives a signal output from the AC generator 21 and transmits ultrasonic waves to the body.

Then, the ultrasonic receiver 24 receives the ultrasonic waves reflected by the body and converts the received ultrasonic waves into an AC signal.

The signal modulator 25 serves to obtain a Doppler signal by modulating the AC signal converted from the reflected ultrasonic waves by the ultrasonic receiver 24.

The signal processor 26 receives the Doppler signal from the signal modulator 25, digitizes the received signal, processes the digitized signal using the algorithm of Fourier transform, and calculates the change in blood flow velocity. In this case, the signal processor 26 calculates a frequency spectrum and, since the frequency in the generated frequency spectrum has a linear relationship with the blood flow velocity, the frequency spectrum represents the distribution of the blood flow velocity.

The controller 30 connected to the signal processor 26 receives a signal from the signal processor 26, stores the calculated blood flow velocity, and displays the calculated blood flow velocity on the screen of the display 40.

As such, when the blood flowmeter 20 using the Doppler effect is used, it is possible to non-invasively and continuously measure the blood flow velocity in real time and simultaneously measure the change in pulse.

Next, a method of performing the test using the cold reflex tester of the present invention will be described.

First, the measurement unit (or ultrasonic probe) of the blood flowmeter 20 that transmits ultrasonic waves to a subject's body and receives ultrasonic waves reflected from the subject's body is brought into contact with the peripheral circulation such as hand or foot of the subject, and then the blood flow velocity is continuously measured and recorded. At this time, the controller 30 stores the measurement result of the blood flow velocity obtained from the blood flowmeter 20 and displays the measurement result on the screen of the display 40.

Then, when the subject brings his or her hand into contact with the contact region 17 of the cold stimulator 10 that maintains a fixed low temperature, the controller 30 receives an electrical signal from the touch sensor 19 of the cold stimulator 10 and thus recognizes the start time at which a cold stimulus is applied. The controller 30 may display the start time at which the cold stimulus is applied on the screen of the display 40.

At this time, the blood flow velocity is continuously measured by the blood flowmeter 20, and the controller 30 records the time period during which the subject's body is in contact with the contact region 17 of the cold stimulator 10 and continuously stores and displays the measurement result of the blood flow velocity obtained from the blood flowmeter 20 on the screen of the display 40.

Subsequently, when the subject removes his or her hand from the cold stimulator 10 after a predetermined time, the controller 30 receives a signal indicating the release of the touch from the touch sensor 19 and records the stimulation end time at which the subject's hand is removed from the touch sensor 19. The controller 30 may display the stimulation end time at which the cold stimulus is taken away on the screen of the display 40.

Then, even after the subject's hand is removed from the cold stimulator 10, the blood flow velocity is continuously measured by the blood flowmeter 20. At this time, the controller 30 continuously stores the measurement result after the subject's hand is removed from the cold stimulator 10, displays the measurement result on the screen of the display 40, and records the recovery time from the measurement result.

FIG. 4 is a graph showing a change in blood flow velocity measured by the blood flowmeter in the cold reflex tester in accordance with the present invention. As shown in FIG. 4, the blood flow velocity decreases at the time (stimulation start time) at which the subject brings his or her body into contact with the cold stimulator, and the decreased blood flow velocity is maintained within a predetermined range while the cold stimulus is applied to the subject's body.

Moreover, the blood flow velocity increases at the time (stimulation end time) at which the subject's body is removed from the cold stimulator, and the blood flow velocity is returned to its original value after a certain time elapses.

As described above, the cold reflex tester of the present invention has the following advantages. First, since an electrical device capable of controlling the temperature and maintaining a fixed temperature at a low level based on an electrical signal from a controller is used in a cold stimulator that is brought into contact with a subject's body as a means for applying a cold stimulus to the subject's body, it is possible to apply a more constant stimulus to the subject's body during measurement and thus improve the reliability of the measurement.

Moreover, it is possible to easily monitor the body's reaction in real time by measuring the blood flow velocity of the subject using a blood flowmeter to detect the body's reaction against the cold stimulus.

Especially, a blood flowmeter using the Doppler effect can non-invasively measure the blood flow velocity in real time and simultaneously measure the change in pulse.

Furthermore, since a touch sensor is mounted on the surface of the cold stimulator that is brought into contact with the subject's body, it is possible to detect stimulation start and end times from a signal from the touch sensor.

In addition, a considerable portion of the measurement process including applying the cold stimulus to the subject's body and obtaining a measurement result from the blood flowmeter can be automated.

As above, preferred embodiments of the present invention have been described and illustrated, however, the present invention is not limited thereto, rather, it should be understood that various modifications and variations of the present invention can be made thereto by those skilled in the art without departing from the spirit and the technical scope of the present invention as defined by the appended claims.

1. A cold reflex tester, which applies a cold stimulus to a subject's body and detects and monitors the body's reaction against the cold stimulus, the cold reflex tester comprising:
a cold stimulator for applying a cold stimulus to the subject's body being in contact with the cold stimulator by maintaining a fixed low temperature based on an electrical signal applied from a controller;
a blood flowmeter for measuring blood flow velocity of the subject to detect the body's reaction against the cold stimulus;
the controller for controlling the entire system including the cold stimulator and the blood flowmeter and obtaining a measurement result from the blood flowmeter; and
a display for displaying the measurement result received from the controller on a screen.

2. The cold reflex tester of claim 1, wherein the cold stimulator comprises a Peltier module controlled by an electrical signal applied from the controller.

3. The cold reflex tester of claim 2, wherein the cold stimulator comprises a heat sink mounted on a high temperature region of the Peltier module, a cooling fan for cooling the heat sink, and a contact region, with which the subject's body is brought into contact, formed on a low temperature region of the Peltier module.

4. The cold reflex tester of claim 1, further comprising a touch sensor mounted on the surface of the cold stimulator to detect a touch of the body and a release of the touch and output an electrical signal to the controller.

5. The cold reflex tester of claim 4, wherein the controller recognizes and records stimulation start and end times based on the electrical signal input from the touch sensor.

6. The cold reflex tester of claim 5, wherein the controller displays the stimulation start and end times on the display.

7. The cold reflex tester of claim 1, wherein a temperature sensor is provided in the cold stimulator such that the controller receives a signal of the temperature sensor to control the operation of the cold stimulator.

8. The cold reflex tester of claim 1, wherein the blood flowmeter is a blood flowmeter using the Doppler effect that measures blood flow velocity by transmitting ultrasonic waves to the body and detecting the frequency of the ultrasonic waves reflected from blood vessels.

9. The cold reflex tester of claim 2, wherein a temperature sensor is provided in the cold stimulator such that the controller receives a signal of the temperature sensor to control the operation of the cold stimulator.
